(19) United States
(12) Patent Application Publication
(10) Pub. No.: US 2018/0371519 A1
(43) Pub. Date: Dec. 27, 2018

(54) METHOD FOR IDENTIFICATION OF SIMILAR SPECIES USING NEGATIVE MARKER, AND APPARATUS FOR THE SAME

(71) Applicant: AMIT, INC., Seongnam-si (KR)

(72) Inventors: Jongseo LEE, Suwon-si (KR); Songkuk KIM, Seongnam-si (KR); Eung Joon JO, Old Tappan, NJ (US)

(21) Appl. No.: 16/015,329

(22) Filed: Jun. 22, 2018

Related U.S. Application Data
(60) Provisional application No. 62/524,023, filed on Jun. 23, 2017.

(51) Int. Cl. C12Q 1/04; G06N 99/00

(52) U.S. Cl. CPC C12Q 1/04; G01N 2560/00; G06N 99/005

(57) ABSTRACT

The present disclosure relates to a method and apparatus for identification of similar species, and more particularly to method and apparatus for identification of similar species based on machine learning using negative markers. According to an aspect of the present disclosure, a method for identifying similar species may comprise: extracting first mass information for an input sample; classifying the input sample using a machine learning model based on at least a negative marker, based on the first mass information; and identifying a species for the input sample based on the classification result.

### CROSS-REFERENCE TO RELATED APPLICATIONS

The present application claims priority to U.S. Provisional Patent Application No. 62/524,023 filed on Jun. 23, 2017, which is hereby incorporated by reference in its entirety.

### TECHNICAL FIELD

The present disclosure relates to a method and apparatus for identification of similar species, and more particularly to method and apparatus for identification of similar species based on machine learning using negative markers.

### BACKGROUND ART

Mass spectrometry is widely used to identify the mass composition of an object. For example, a microorganism may be identified by applying markers selected based on the extracted mass information for an unknown microorganism. Markers are characteristics that can be used to uniquely identifying a microorganism. In addition, the microorganism identification performance can be improved by combining the extracted mass composition information and the machine learning techniques.

Even with the mass spectrometry, it is difficult to accurately identify or distinguish similar microorganism species through conventional methods, since the mass spectral patterns of similar microorganism species are very similar to each other. Therefore, it is required to improve identification performance among similar species. DISCLOSURE

### Technical Problem

It is a technical object of the present invention to provide a method and apparatus for improving identification performance among similar species.

It is an additional technical object of the present invention to provide a method and apparatus for improving microorganism identification performance regardless of machine learning scheme.

It is an additional technical object of the present invention a method and apparatus for classifying microorganism by applying negative markers to various machine learning schemes.

The technical objects to be achieved by the present disclosure are not limited to the technical matters mentioned above, and other technical objects not mentioned are to be clearly understood by those skilled in the art from the following description.

### Technical Solution

According to an aspect of the present disclosure, a method for identifying similar species may comprise: extracting first mass information for an input sample; classifying the input sample using a machine learning model based on at least a negative marker, based on the first mass information; and identifying a species for the input sample based on the classification result.

According to an additional aspect of the present disclosure, an apparatus for identifying similar species may comprise: a mass analyzer for extracting first mass information for an input sample; and a classifier for classifying the input samples using a machine learning model based on at least a negative marker stored in a negative marker database, based on the first mass information, wherein the apparatus identifies a species for the input sample based on the classification result.

In the various aspects of the present disclosure, the input sample may be classified using a positive marker and the negative marker.

In the various aspects of the present disclosure, each of the positive marker and the negative marker may be previously extracted for each of samples belonging to the similar species.

In the various aspects of the present disclosure, the positive marker may comprise mass information that frequently appears in a target species compared to an opposition species.

In the various aspects of the present disclosure, the negative marker may comprise mass information that frequently appears in an opposition species compared to a target species.

In the various aspects of the present disclosure, each of the positive marker and the negative marker may be extracted based on a bin set for a mass spectrum for each of the samples belonging to the similar species.

In the various aspects of the present disclosure, each of the positive marker and the negative marker may be expressed by a set of number of the bin in which a peak value of the mass spectrum is located.

In the various aspects of the present disclosure, a bin may partially overlap one or more other bins.

In the various aspects of the present disclosure, each of the positive marker and the negative marker may be calculated based on frequency information of a bin in which a peak value of the mass spectrum is located.

In the various aspects of the present disclosure, each of the positive marker and the negative marker may be extracted based on a Term Frequency-Inverse Document Frequency (TF-IDF) calculation for the frequency information of the bin.

In the various aspects of the present disclosure, the positive marker may be calculated based on a math expression

$$TF - {IDF}_{bin ( i )} = \frac{{F}_{bin ( i ) , {sample}_{t}}}{{N}_{t}} \times log ( \frac{{N}_{o}}{{F}_{bin ( i ) , {sample}_{o}}} ) ,$$

where t denotes a target species, o denotes an opposition species, Nt denotes a total number for the target species, No denotes a total number for the opposition species, and Fbin(i) denotes a count value for the i-th bin.

In the various aspects of the present disclosure, the positive marker may be set when the TF-IDF value calculated by the math expression exceeds a predetermined threshold value.

In the various aspects of the present disclosure, the negative marker may be calculated based on a math expression

$$TF - {IDF}_{bin ( i )} = \frac{{F}_{bin ( i ) , {sample}_{o}}}{{N}_{o}} \times log ( \frac{{N}_{t}}{{F}_{bin ( i ) , {sample}_{t}}} )$$

where t denotes a target species, o denotes an opposition species, Nt denotes a total number for the target species, No denotes a total number for opposition species, and . Fbin(i) denotes a count value for the i-th bin.

In the various aspects of the present disclosure, the negative marker may be set when the TF-IDF value calculated by the math expression exceeds a predetermined threshold value.

In the various aspects of the present disclosure, each of the positive marker and the negative marker may be generated as a preprocessing for extracting features for learning of the machine learning model.

In the various aspects of the present disclosure, the classifying may further comprise calculating a Composite Correlation Index (CCI) based on the first mass information and second mass information previously stored for each of one or more samples; and determining a candidate for the classification based on the calculated CCI.

It is to be understood that the foregoing summarized features are exemplary aspects of the following detailed description of the present disclosure and are not intended to limit the scope of the present disclosure.

### Advantageous Effects

According to the present disclosure, a method and apparatus for improving identification performance among similar species using negative markers related to mass spectrometry may be provided.

According to the present disclosure, a method and apparatus for improving microorganism identification performance using negative markers regardless of machine learning schemes may be provided.

According to the present disclosure, a method and apparatus for improving microorganism identification performance based on machine learning by applying preprocessing for extracting features may be provided.

The advantages of the present disclosure are not limited to the foregoing descriptions, and additional advantages will become apparent to those having ordinary skill in the pertinent art to the present disclosure based upon the following descriptions.

### DESCRIPTION OF DRAWINGS

FIG. 1 is a diagram for illustrating a process for extracting markers according to the present disclosure.

FIG. 2 is a diagram for illustrating a bin scheme used for marker extraction according to the present disclosure.

FIG. 3 is a diagram showing examples of data stored in the positive marker DB and the negative marker DB according to the present disclosure.

FIG. 4 is a diagram showing a process framework for classification of similar species according to the present disclosure.

FIG. 5 is a diagram for illustrating a machine learning model for a similar species classification according to the present disclosure.

FIG. 6 is a diagram illustrating a machine learning process for computing a confusion matrix for a similar species according to the present disclosure.

FIGS. 7 and 8 are diagrams illustrating the results of evaluation metrics for marker-based identification results in accordance with the present disclosure.

FIG. 9 is a diagram illustrating a similar species identification method according to the present disclosure.

### BEST MODE

Hereinafter, embodiments of the present invention will be described in detail so that those skilled in the art can easily carry out the present invention referring to the accompanying drawings. However, the present disclosure may be embodied in many different forms and is not limited to the embodiments described herein.

In the following description of the embodiments of the present invention, a detailed description of known functions and configurations incorporated herein will be omitted when it may make the subject matter of the present disclosure unclear. Parts not related to the description of the present disclosure in the drawings are omitted, and similar parts are denoted by similar reference numerals.

In the present disclosure, when an element is referred to as being “connected”, “coupled”, or “connected” to another element, it is understood to include not only a direct connection relationship but also an indirect connection relationship. Also, when an element is referred to as “containing” or “having” another element, it means not only excluding another element but also further including another element.

In the present disclosure, the terms “first”, “second”, and so on are used only for the purpose of distinguishing one element from another, and do not limit the order or importance of the elements unless specifically mentioned. Thus, within the scope of this disclosure, the first component in one embodiment may be referred to as a second component in another embodiment, and similarly a second component in one embodiment may be referred to as a second component in another embodiment.

In the present disclosure, components that are distinguished from one another are intended to clearly illustrate each feature and do not necessarily mean that components are separate. That is, a plurality of components may be integrated into one hardware or software unit, or a single component may be distributed into a plurality of hardware or software units. Accordingly, such integrated or distributed embodiments are also included within the scope of the present disclosure, unless otherwise noted.

In the present disclosure, the components described in the various embodiments do not necessarily mean essential components, but some may be optional components. Accordingly, embodiments consisting of a subset of the components described in one embodiment are also included within the scope of this disclosure. Also, embodiments that include other components in addition to the components described in the various embodiments are also included in the scope of the present disclosure.

The definitions of the terms used in the present disclosure are as follows.

Marker: Features used for uniquely identifying a target

Positive marker: Features that appears more frequently in target species than in opposition species

Negative marker: Features that appears more frequently in opposition species than in target species

Bin: Specific interval of a spectrum

The definitions of the abbreviations used in the present disclosure are as follows.

MALDI-TOF: Matrix-Assisted Laser Desorption/Ionization-Time-Of-Flight

MS: Mass Spectrometry

CCI: Composite Correlation Index

TF-IDF: Term Frequency-Inverse Document Frequency

Hereinafter, a method and apparatus for identifying similar species using negative markers according to the present disclosure will be described.

MALDI-TOF MS is widely used because it can identify microorganisms at high speed based on protein mass composition. Microorganisms may be identified by selecting markers that distinguish the microorganism from other species based on extracted mass composition information for a certain microorganism. By combining mass information extracted by a method such as MALDI-TOF MS with machine learning scheme, the performance of microorganism classification may be improved.

Classification of microorganisms is challenging especially in the case of mycobacteria, and is very important. This is because some microorganism species show similar mass composition, but different pathogens must be treated with different antibiotics. Since the MALDI-TOF mass spectrometric patterns of similar microorganism species are very similar to each other, it is difficult to accurately identify similar microorganism species through conventional methods. For example, in the case of mycobacterium tuberculosis, the mass spectrometric patterns between species are very similar to each other, and the accuracy of identification is relatively low compared to other bacteria. Although the components of each microorganism species are very similar to each other, the prescription for the patient must be different for each species, thus classification for similar microorganism species is very important. In addition, CCI is an efficient method for finding similar bacteria based on mass spectrometry, but cannot accurately classify similar species such as the mycobacterium abscessus group. Accordingly, there is a need for a new scheme for identifying or classifying microorganisms different from conventional schemes.

According to the present disclosure, microorganism identification performance may be improved by using negative markers. Further, according to the present disclosure, by applying a new machine learning scheme using positive markers and negative markers, identification and classification performance in the mass spectrometry of microorganism may be enhanced. Further, the present disclosure also provides a new scheme of applying preprocessing to features used in machine learning. For example, preprocessing for features includes extracting negative markers. In addition, preprocessing for features includes extracting positive markers and negative markers separately. Accordingly, even when any machine learning scheme is applied, identification performance of similar species may be enhanced. That is, regardless of the machine learning schemes, the performance of identification and classification of microorganisms may be enhanced.

In the present disclosure, the identification or classification of subtypes or subspecies of the mycobacterium abscessus group and the mycobacterium fortuitum group is described as a representative example However, the scope of the present disclosure is not limited thereto, and includes identification or classification schemes using negative markers for similar species of various microorganisms.

Also, in the present disclosure, a support vector machine (SVM) is described as a representative example of a machine learning scheme. However, the scope of the present disclosure is not limited thereto, and includes applying similar species identification or classification schemes using negative markers according to the present disclosure to various machine learning schemes such as k-nearest neighbor (k-NN), neural network, random forest algorithm.

Hereinafter, extracting positive markers and negative markers will be described first, and a model for classifying similar species using the extracted markers will be described.

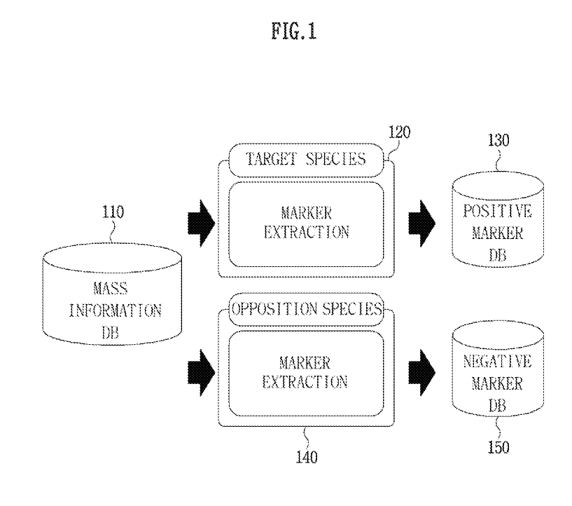

FIG. 1 is a diagram for illustrating a process for extracting markers according to the present disclosure.

The present disclosure includes a new framework for extracting positive and negative markers from each subtype of mycobacteria and using them as a machine learning model. By using such positive and negative markers, the model according to the present disclosure may greatly improve the accuracy of classifying subspecies in any type of machine learning.

In FIG. 1, the mass information database (DB) 110 may include a dataset of mass information for species belonging to one or more microorganism groups. Specifically, the mass information DB 110 may include mass information for each of one or more species belonging to each of one or more microorganism groups. For example, mass information may be obtained by MALDI-TOF MS analysis for each of microorganism samples.

Table 1 shows an example of statistics for a dataset included in the mass information DB 110.

TABLE 1

| Group | Species | Number of spectra |
|---|---|---|
| M. abscessus | M. abscessus | 167 |
| | M. bolletii | 95 |
| | M. massiliense | 163 |
| M. fortuitum | M. fortuitum | 124 |
| | M. conceptionense | 109 |
| | M. neworleansense | 18 |
| | M. peregrinum | 58 |
| | M. porcinum | 62 |

Table 1 shows that M. abscessus, M. bolletii, and M. massiliense belong to the M. abscessus group, and the number of mass spectra for each sample is 167, 95, and 163. In addition, M. fortuitum, M. conceptionense, M. neworleansense, M. peregrinum and M. porcinum belong to M. fortuitum group, and the number of mass spectra for each sample is 124, 109, 18, 58 and 62. It is assumed that the mass information DB 110 includes actual mass spectrum information for each species.

In the marker extraction process 120 for a target species of FIG. 1, a marker may be extracted based on mass information for a specific target species among data included in the mass information DB 110. For example, a positive marker may include mass information that frequently appears in a target species compared to other similar species (e.g., opposition species). The result of the marker extraction 120 may be stored and maintained in the positive marker DB 130.

In the marker extraction process 140 for an opposition species in FIG. 1, the marker can be extracted based on mass information for a specific opposition species among data included in the mass information DB 110. For example, a negative marker may include mass information that frequently appears in opposition species compared to the target species. The result of the marker extraction 140 may be stored and maintained in the negative marker DB 150.

For example, M. abscessus, M. bolletii and M. massiliense are similar groups. When the selected target is M. abscessus, M bolletii and M. massiliense may be opposition species.

As such, markers representing features of a specific bacterium may be extracted from a mycobacterial dataset. The TF-IDF scheme may be applied as an example of marker extraction, which will be described later.

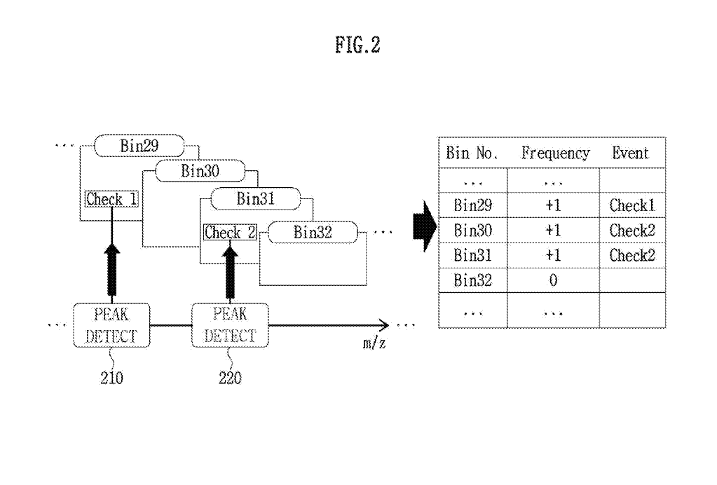

FIG. 2 is a diagram for illustrating a bin scheme used for marker extraction according to the present disclosure.

MALDI-TOF MS does not necessarily produce the same results even if the same experiments are repeated. Even for the same molecule, the total flight time may vary slightly depending on the angle of ion flight. This may cause a peak shift in the mass spectrum.

To consider the peak shift, binning may be applied for mass and bin windows may partially overlap as shown in FIG. 2.

By calculating the frequency of the bin in which the peak value is located in the mass spectrum of a sample, the features of the mass spectrum of the sample may be expressed as an aggregation of bin numbers. Thus, the feature value for a specific sample may be extracted more accurately.

Thus, according to the present disclosure, data preprocessing that applies bins to mass information is applied. Thus, the effects of observation errors such as peak shift may be reduced.

Specifically, the mass information stored in each of the positive marker DB 130 and the negative marker DB 150 may include a set of mass bin numbers. A mass bin may correspond to a certain section in the mass spectrum. In addition, a mass bin may partially overlap with one or more other mass bins.

For example, it is assumed that the entire range of the mass spectrum is covered by 100 bins of the same size. Bin numbers may be assigned in order such as bin1, bin2, bin3, . . . , bin100 starting with the lower spectral interval. As in the example of FIG. 2, some of the higher mass value portion within bin29 may overlap some of the lower mass value portion within bin30. Also, some of the lower mass value portion within bin30 may overlap some of the higher mass value portion within bin29, and some of the higher mass value portion of bin30 may overlap some of the lower mass value portion of bin31. However, the scope of the present disclosure is not limited to the above-described example, and a certain mass value interval may be set to an interval in which three or more bins overlap, and a certain mass value interval may be covered by only one bin.

In the example of FIG. 2, two peaks 210 and 220 are detected in the signal intensity of the mass-to-charge ratio (m/z) in a portion of the mass spectrum of a specific sample. An event “check1” that the detected peak 210 is confirmed to correspond to bin29 may occur, and an event “check2” that another detected peak 220 is confirmed to correspond to bin30 and also correspond to bin31 may occur. Accordingly, the frequency of bin29 is counted by +1 due to the event of checkl, the frequency of bin30 is counted by +1 due to the event of check2, and the frequency of bin31 is counted by +1 due to the event of check2. Since the peak value is not detected in the interval corresponding to bin32, the frequency of bin32 is counted as zero.

As such, when the original data value belongs to a predetermined interval referred to as a bin, the corresponding data value may be replaced with a representative value of the predetermined interval. The representative value of the interval may generally be a central value of the interval, but is not limited thereto, and a start value, an end value, or any value belonging to the interval may be defined as a representative value. For example, in the example of FIG. 2, the representative value of bin 29 may be given as the number of the bin, that is, 29.

When the size of the bin is large (i.e., the number of bins covering the entire spectral interval is small), the performance to accurately distinguish the sample from other similar species may be degraded. Conversely, when the size of the bin is narrow (i.e., the number of bins covering the entire spectral interval is large), it may be difficult to reduce the effects of observation errors (e.g., peak shift). In view of this, an exemplary size of a bin in the present disclosure may be set to 20 m/z.

In addition, in the range in which the bin windows are overlapped as in the example of FIG. 2, it may be set as the start position and the end position of every even-numbered bins are not overlapped with each other and continuous, and the start position and the end position of every odd-numbered bins are not overlapped with each other and continuous. For example, as shown in FIG. 2, the end position of bin 29 may not be set to overlap with the start position of bin 31, but may be set to cover continuous values.

The scope of the present disclosure is not limited to the above example bin size and overlapping range, and may be appropriately set in consideration of the characteristics of the dataset. That is, the characteristics of the present disclosure is in applying the preprocessing for extracting the positive markers and the negative markers using the set bins, and is not limited to specific values such as the size, number, overlapping range, and the like of bins.

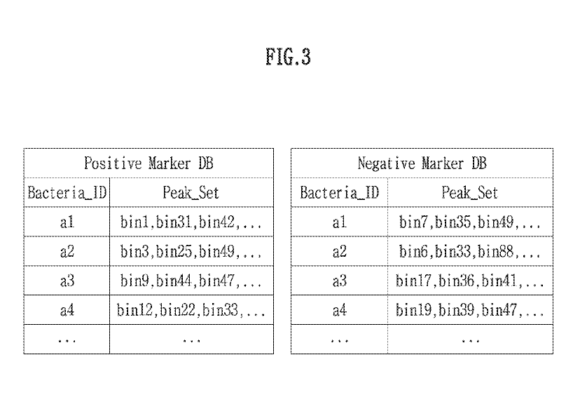

FIG. 3 is a diagram showing examples of data stored in the positive marker DB and the negative marker DB according to the present disclosure.

As shown in FIG. 2, when the mass data features for a sample is stored in the DB in the form of a set of bin numbers, the positive markers and the negative markers may be extracted from the information. That is, by calculating the bin frequency, it is possible to detect which bin(s) frequently appear in the target species or opposition species. By calculating an adjusted TF-IDF for the bin frequency information for each species, it is possible to eventually extract the positive markers and the negative markers. For example, the TF-IDF calculation described below may be applied in marker extraction 120 for target species and marker extraction 130 for opposition species in FIG. 1.

Math FIG. 1 represents a mathematical expression for extracting positive markers.

$$TF - {IDF}_{bin ( i )} = \frac{{F}_{bin ( i ) , {sample}_{t}}}{{N}_{t}} \times log ( \frac{{N}_{o}}{{F}_{bin ( i ) , {sample}_{o}}} ) \qquad [ Math FIG . 1 ]$$

In Math FIG. 1, t denotes a target species and o denotes an opposition species. Nt means the total number for the target species, and No means the total number for opposition species. Fbin(i) denotes a count value for the i-th bin.

In addition, the TF-IDF threshold may be used as a reference for distinguishing positive markers and negative markers. For example, when the bin frequency in the target species is 85% and the bin frequency in the opposition species is 15%, the TF-IDF threshold may be 0.676498. Thus, when the TF-IDF value in each bin exceeds a threshold (e.g., 0.676498), the bin may be set as a positive marker.

Math FIG. 2 represents a mathematical expression for extracting negative markers.

$$TF - {IDF}_{bin ( i )} = \frac{{F}_{bin ( i ) , {sample}_{o}}}{{N}_{o}} \times log ( \frac{{N}_{t}}{{F}_{bin ( i ) , {sample}_{t}}} ) \qquad [ Math . FIG . 2 ]$$

Math FIG. 2 corresponds to Math FIG. 1 by exchanging target species with opposition species. That is, in Math FIG. 2, t denotes a target species and o denotes an opposition species. Nt means the total number for the target species, and No means the total number for opposition species. Fbin(i) denotes a count value for the i-th bin. A meaningful markers may be identified based on the ranking and scale for the TF-IDF results calculated as in Math FIG. 2.

In addition, the TF-IDF threshold may be used as a reference for distinguishing positive markers and negative markers. For example, when the bin frequency in the opposition species is 85% and the bin frequency in the target species is 15%, the TF-IDF threshold may be 0.676498. Thus, when the TF-IDF value in each bin exceeds a threshold (e.g., 0.676498), the bin may be set as a negative marker.

A meaningful markers may be identified based on the ranking and scale for the calculated TF-IDF results calculated as in Math FIGS. 1 and 2. Using this, a positive marker DB and a negative marker DB for each bacteria may be set as shown in FIG. 3.

For example, in FIG. 3, a positive marker for a bacteria with a bacterial identifier (Bacteria_ID) of al includes information on a bin number set (i.e., a set of bin numbers where peaks are detected) of bin1, bin31, bin42, . . . . Further, the negative marker for the bacteria having the same al identifier may include information on a bin number set of bin7, bin35, bin49, In addition, positive and negative markers may also be stored for each bacteria (e.g., a2, a3, a4, . . . ).

As a result of this preprocessing of the dataset, positive and negative markers may be determined, and by analyzing the mass features of an unknown sample using the above preprocessing results (especially using negative markers), it is possible to accurately identify or classify which bacteria the sample corresponds to.

Hereinafter, following the descriptions of extracting the positive markers and negative markers as described above, a description will be given of a model for classifying similar species using the extracted markers.

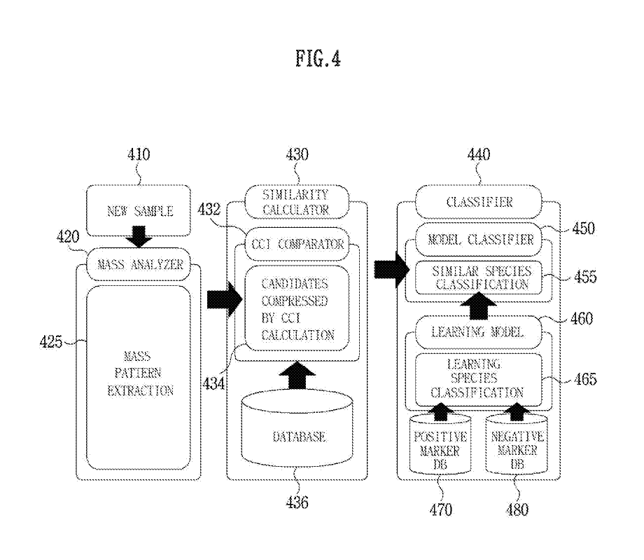

FIG. 4 is a diagram showing a process framework for classification of similar species according to the present disclosure.

When a new sample 410 is input to the similar species classification process, a mass analysis for that sample may be performed in the mass analyzer 420. As a result of the mass analysis, the mass pattern 425 for the sample may be extracted. For example, a mass spectrometry of a sample may be performed using MALDI-TOF, and the mass pattern may be obtained in the form of a mass spectrum. That is, the mass information may include mass and intensity values.

The similarity calculator 430 may calculate the similarity between the extracted mass pattern information 425 for the sample and the information stored in the database 436. For example, the calculation of the similarity may be performed by calculating CCI of the extracted mass pattern information 425 for the input sample and the information stored in the database 436. Specifically, for the mass and intensity values obtained for the input sample 410 and the mass and intensity values previously obtained for the samples stored in the database 436, the similarity between them may be obtained using the CCI calculation.

A similar group may be extracted through CCI calculations, but it is not sufficient to accurately identify the target among similar groups. In order to solve this problem, it is possible to accurately classify similar species in the CCI calculation result by allowing the machine learning model to learn the classification using the negative markers according to the present disclosure. More specifically, according to the present disclosure, by allowing a machine learning model to learn classification using positive markers and negative markers, it is possible to more accurately classify similar species from the CCI calculation results.

For example, the CCI comparator 432 may calculate CCI based on the extracted mass information (i.e., the first mass information) with respect to the input sample 410 and the mass information (i.e., the second mass information) with respect to the samples previously stored in the database 436. Since the database 436 may have previously stored mass information for one or more samples, the CCI calculation may be performed based on the second mass information for each of one or more samples of the database 436. That is, a CCI calculation can be performed for the first mass information and each of the one or more second mass information.

The CCI comparator 432 may determine candidates of samples stored in the database 436 that matches the input sample 410 by calculating a CCI value for the first mass information and each of the one or more second mass information. Information indicating the candidates 434 compressed through the CCI calculation may be transmitted to the classifier 440.

The classifier 440 may perform the classification process using the machine learning model for the candidates 434 compressed through the CCI calculation. The classifier 440 may include a model classifier 450 and a learning model 460. The learning model 460 may perform a learning 465 about classifications for each species using the information stored in the positive marker DB 470 and the information stored in the negative marker DB 480 as feature values. The model classifier 450 may perform a similar species classification 455 for the new sample 410 based on the learning model 460 and as a result a specific class may be derive. The derived result may be used again as a sample of machine learning.

As such, when a data value for a new sample is entered into a machine learning classifier, a specific class may be derived based on a pre-learned model. Also, based on the classification result, the species for the input new sample input may be identified.

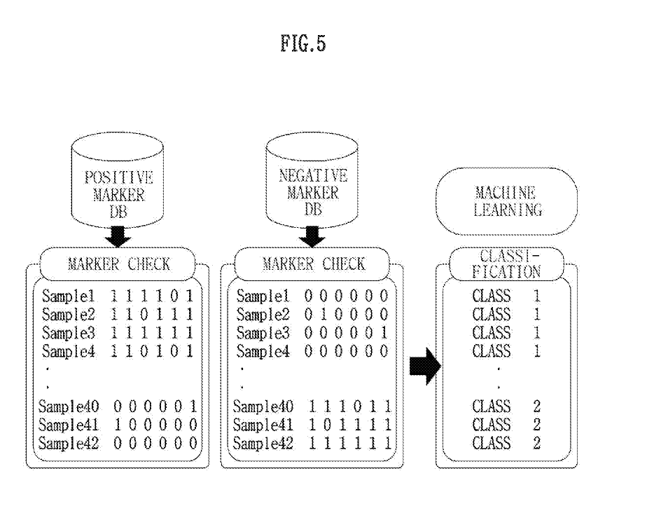

FIG. 5 is a diagram for illustrating a machine learning model for a similar species classification according to the present disclosure.

FIG. 5 shows and an example of a machine learning process using positive and negative markers as features.

As described above, the positive markers may include mass information for the target species, and the negative markers may include mass information for the opposition species. For each sample, the mass bin information may be evaluated. For example, the evaluation of the mass bin information may be performed using a Boolean operator.

In the example of FIG. 5, the positive marker check result for sample 1 is denoted by 111101, and the negative marker check result is denoted by 000000. Here, 1 means true and 0 means false. Accordingly, it may be learned that the sample 1 is classified into class 1. Likewise, in the case of samples 2 to 4 including a check result in which the positive marker check result is relatively more matched than the negative marker check result, the sample may be learned to be classified as class 1. On the other hand, in the case of samples 40 to 42 including a check result in which the positive marker check result is relatively less matched than the negative marker check result, the samples may be learned to be classified into class 2.

As shown in the example of FIG. 5, there is a clear difference between the target species and opposition species. As described above, the performance of the classifier based on the machine learning model may be greatly improved by using the positive markers and the negative markers.

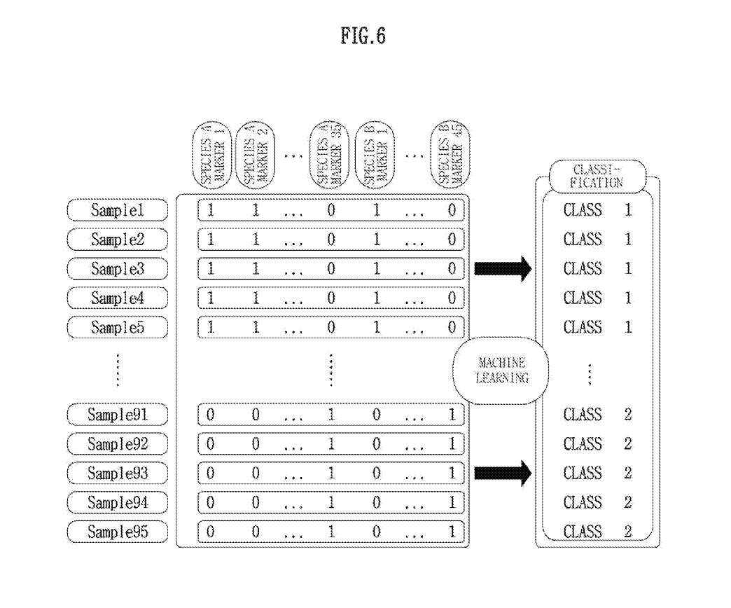

FIG. 6 is a diagram illustrating a machine learning process for computing a confusion matrix for a similar species according to the present disclosure.

In the example of FIG. 6, a check result for a marker 1 of species A, a marker 2 of species A, . . . , a marker 35 of species A, a marker 1 of species B, . . . , a marker 45 of species B is shown as 11 . . . 01 . . . 0. Next, for each of the sample 2 to the sample 95, the check results from the marker 1 of species A to the marker 45 of the species B are exemplarily shown. Based on the result of the marker check, the machine learning model may classify each of the samples into Class 1, Class 2, . . . and the like, and such classification result may be learned.

In addition, in the example of FIG. 6, the check results of Samples 1 to 95 are shown as 11111 . . . 00000 for the marker 1 of species A. Further, FIG. 6 exemplarily shows the check results of the samples 1 to 95 for each of the marker 2 of species A to marker 45 of species B.

As such, in the example of FIG. 6, the species has a Boolean vector from positive markers and negative markers. These vectors may be used in machine learning models for computing confusion matrices.

By learning the every markers for a similar species, it is possible to classify the samples more accurately based on the machine learning model. By computing a confusion matrix, a specific entry may be identified for different groups (e.g., different species). In addition, by calculating the confusion matrix, the standard error for the model using the positive markers and the negative markers according to the present disclosure may be assessed, and by assessing the rate (i.e., percentage) of accurate identification of the species, an internal stability may be measured. Such calculation of the confusion matrix may be applied for various machine learning schemes such as SVM, k-NN, neural network, and random forest.

As for evaluation metrics, two schemes may be applied.

The first is a scheme using precision, recall and f-score, and the second is a scheme using accuracy.

The precision, recall and f-score may be defined by Math FIG. 3.

$$Precision = \frac{tp}{tp + fp} \\ Recall = \frac{tp}{tp + fn} \\ f \text{-} score = \frac{2 \times precision \times recall}{precision + recall} \qquad [ Math FIG . 3 ]$$

In Math FIG. 3, tp means true positive, fp means false positive, and fn means false negative. Also, the f-score corresponds to the harmonic mean of the precision and the recall.

The accuracy may be defined by Math FIG. 4.

$$Accuracy = \frac{tp + tn}{tp + fp + fn + tn} \qquad [ Math FIG . 4 ]$$

In Math FIG. 3, tp means true positive, fp means false positive, to means true negative, and fn means false negative.

Tables 2 and 3 below show a multi-class confusion matrices including the results of similar species identification for the test set as shown in Table 1.

TABLE 2

| Marker-based SVM model | | | |
|---|---|---|---|
| (M. abscessus group) | P1 | P2 | P3 |
| T1 | 94.05% | 4.17% | 1.79% |
| T2 | 15.42% | 77.71% | 6.88% |
| T3 | 0.73% | 1.83% | 97.44% |

Table 2 shows the identification results of the marker-based SVM model for the M. abscessus group. T means the correct species, and P means the predicted species. Indices 1, 2 and 3 refer to M. abscessus, M. bolletii and M. massiliense, respectively.

TABLE 3

| Marker-based | | | | | |
|---|---|---|---|---|---|
| SVM model | | | | | |
| (M. fortuitum | | | | | |
| group) | P1 | P2 | P3 | P4 | P5 |
| T1 | 100% | 0% | 0% | 0% | 0% |
| T2 | 0% | 100% | 0% | 0% | 0% |
| T3 | 1.11% | 0% | 82.22% | 15.56% | 1.11% |
| T4 | 1.03% | 0% | 0% | 98.62% | 0.34% |
| T5 | 0.32% | 0% | 0% | 1.94% | 97.74% |

Table 3 shows the identification results of the marker-based SVM model for the M. fortuitum group. T means the correct species, and P means the predicted species. Indices 1, 2, 3, 4 and 5 refer to M. fortuitum, M. conceptionense, M. neworleansense, M. peregrinum and M. porcinum, respectively.

Table 2 and Table 3 all show highly accurate species discrimination results. Table 2 shows that predicting M. bolletii is difficult compared to predicting other species, and Table 3 shows that T3 shows a lack of samples to learn the pattern, but shows very high classification performance when the samples are sufficient. This pattern is also observed for other learning models as shown in Tables 4 to 9 below.

Tables 4, 6 and 8 below show the identification results of the marker-based machine learning model (k-NN, neural network, random forest model, respectively) for the M. abscessus group as shown in Table 2, and Tables 5, 7 and 9 show the identification results of the marker-based machine learning model (k-NN, neural network, random forest model, respectively) for the M. fortuitum group.

TABLE 4

| Marker-based k-NN model | | | |
|---|---|---|---|
| (M. abscessus group) | P1 | P2 | P3 |
| T1 | 93.45% | 5.00% | 1.55% |
| T2 | 15.42% | 69.38% | 15.21% |
| T3 | 0.24% | 0.73% | 99.02% |

TABLE 5

| Marker- | | | | | |
|---|---|---|---|---|---|
| based | | | | | |
| k-NN | | | | | |
| model | | | | | |
| (M. | | | | | |
| fortuitum | | | | | |
| group) | P1 | P2 | P3 | P4 | P5 |
| T1 | 99.03% | 0.97% | 0% | 0% | 0% |
| T2 | 0% | 100% | 0% | 0% | 0% |
| T3 | 2.22% | 0% | 87.78% | 7.78% | 2.22% |
| T4 | 2.76% | 0% | 0% | 95.86% | 1.38% |
| T5 | 0% | 0.32% | 0% | 0% | 99.68% |

TABLE 6

| Marker-based neural | | | |
|---|---|---|---|
| network model | | | |
| (M. abscessus group) | P1 | P2 | P3 |
| T1 | 88.21% | 10.60% | 1.19% |
| T2 | 22.50% | 64.79% | 12.71% |
| T3 | 0.49% | 10.98% | 88.54% |

TABLE 7

| Marker- | | | | | |
|---|---|---|---|---|---|
| based neural | | | | | |
| network model | | | | | |
| (M. fortuitum | | | | | |
| group) | P1 | P2 | P3 | P4 | P5 |
| T1 | 93.51% | 5.97% | 0.32% | 0% | 0% |
| T2 | 4.18% | 85.82% | 8.91% | 1.09% | 0% |
| T3 | 0% | 15.56% | 46.67% | 28.89% | 8.89% |
| T4 | 0.69% | 2.76% | 21.38% | 63.45% | 11.72% |
| T5 | 0% | 0% | 0.65% | 9.03% | 90.32% |

TABLE 8

| Marker-based random | | | |
|---|---|---|---|
| forest model | | | |
| (M. abscessus group) | P1 | P2 | P3 |
| T1 | 92.38% | 5.00% | 2.62% |
| T2 | 13.96% | 76.04% | 10.00% |
| T3 | 1.22% | 1.83% | 96.95% |

TABLE 9

| Marker-based | | | | | |
|---|---|---|---|---|---|
| random forest model | | | | | |
| (M. fortuitum group) | P1 | P2 | P3 | P4 | P5 |
| T1 | 98.71% | 1.29% | 0% | 0% | 0% |
| T2 | 0% | 100% | 0% | 0% | 0% |
| T3 | 6.67% | 0% | 85.56% | 6.67% | 1.11% |
| T4 | 1.72% | 0% | 0% | 98.28% | 0% |
| T5 | 0% | 0% | 0% | 0% | 100% |

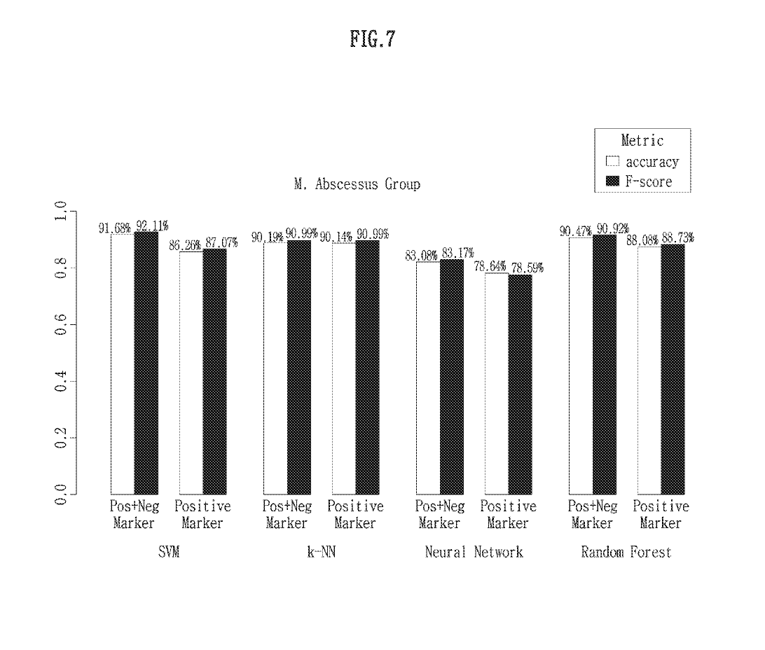

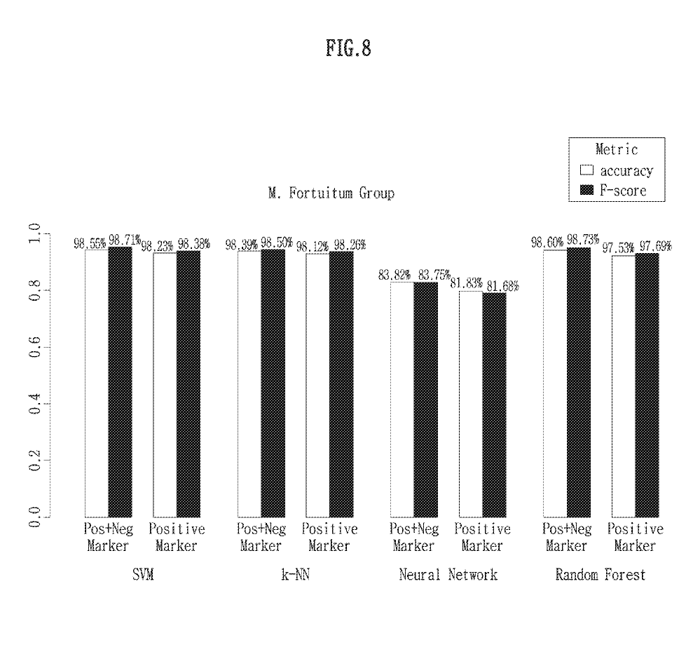

FIGS. 7 and 8 are diagrams illustrating the results of evaluation metrics for marker-based identification results in accordance with the present disclosure.

FIG. 7 shows the accuracy and the f-score values for each machine learning schemes for the identification results using both the positive markers and the negative markers and the identification results using only the positive marker, for the M. abscessus group.

FIG. 7 shows the accuracy and the f-score values for each machine learning schemes for the identification results using both the positive markers and the negative markers and the identification results using only the positive marker, for the M. fortuitum group.

As shown in FIGS. 7 and 8, compared to a conventional machine learning model using only positive markers, the accuracy is improved by approximately 1 to 5% in a machine learning model using positive markers and negative markers according to the present disclosure. Thus, the similar species identification scheme using the negative markers according to the present disclosure may improve the similar species identification performance regardless of the machine learning schemes.

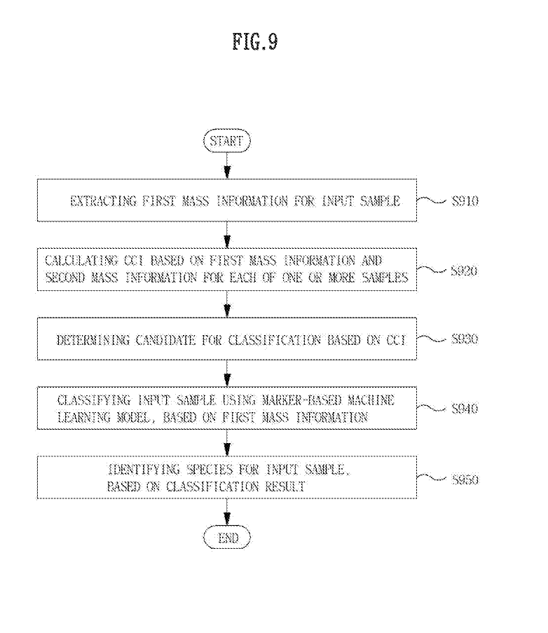

FIG. 9 is a diagram illustrating a similar species identification method according to the present disclosure.

In step S910, the first mass information for the input sample may be extracted. For example, based on the MALDI-TOF MS, mass spectrum or mass pattern information for the input sample may be extracted.

In step S920, the CCI may be calculated based on the first mass information extracted in step S910 and the second mass information previously stored for each of the one or more samples. The second mass information may be previously extracted for the one or more samples and stored in a database.

In step S930, the candidate for the classification may be determined based on the CCI calculation result of step S920.

The steps S920 and S930 may lower the complexity of the similar species classification using the marker-based machine learning model and improve the performance in terms of determining the candidates of the similar species classification. The scope of the present disclosure also includes a case where the steps S920 and S930 are not performed, and the input samples may be sufficiently classified among similar species by using a marker-based machine learning model based on the first mass information.

In step S940, based on the first mass information extracted in step S910, the input sample may be classified using the marker-based machine learning model. The marker-based machine learning model may include a machine learning model using at least a negative marker. In addition, the marker-based machine learning model may include a machine learning model using positive markers and negative markers.

Each of the positive markers and the negative markers may be extracted in advance for each of the samples belonging to the similar species. For example, each of the positive marker and the negative markers may be extracted based on the bins set for the mass spectrum for each of the samples belonging to the similar species. Thus, extracting the positive markers and the negative markers by applying bins to the mass information of the samples may be performed as a preprocessing for extracting features for learning of the machine learning model.

In step S950, based on the classification results in step S940, the species for the input sample may be identified.

The examples of the present disclosure have described approaches to accurately classifying clinically important mycobacteria. However, the scope of the present disclosure is not limited thereto, and a machine learning scheme using at least negative markers according to the present disclosure may be used for various purposes to classify samples among similar groups. That is, the features for extracting positive markers and negative markers according to the present disclosure, and features for machine learning classifiers based on positive markers and negative markers, may be applied to various technologies for accurately classifying samples among similar groups.

According to the present disclosure, positive markers and negative markers are extracted by the TF-IDF scheme and used as features of machine learning, and particularly, by applying negative markers to a similar species classification and species identification, the classification performance of various machine learning schemes regardless of specific machine learning schemes. Also, according to the present disclosure, by combining the CCI calculation with the marker-based machine learning classifier for the similar species classification, it is possible to more accurately classify similar species that could not be correctly classified by the CCI calculation alone.

Although the exemplary methods of this disclosure are represented by a series of steps for clarity of explanation, they are not intended to limit the order in which the steps are performed, and if necessary, each step may be performed simultaneously or in a different order. In order to implement the method according to the present disclosure, it is possible to include other steps to the illustrative steps additionally, exclude some steps and include remaining steps, or exclude some steps and include additional steps.

The various embodiments of the disclosure are not intended to be exhaustive of all possible combination, but rather to illustrate representative aspects of the disclosure, and the features described in the various embodiments may be applied independently or in a combination of two or more.

In addition, various embodiments of the present disclosure may be implemented by hardware, firmware, software, or a combination thereof. A case of hardware implementation may be performed by one or more application specific integrated circuits (ASICs), digital signal processors (DSPs), digital signal processing devices (DSPDs), programmable logic devices (PLDs), field programmable gate arrays (FPGAs), a general processor, a controller, a microcontroller, a microprocessor, and the like.

The scope of the present disclosure is to encompass software or machine-executable instructions (e.g., operating system, applications, firmware, instructions, and the like) by which operations according to method of various embodiments are executed on a device or a computer, and non-transitory computer-readable media executable on the device or the computer, on which such software or instructions are stored.

### INDUSTRIAL APPLICABILITY

Embodiments of the present disclosure may be applied to various analysis methods and apparatuses based on machine learning.

1. A method for identifying similar species, the method comprising:
extracting first mass information for an input sample;
classifying the input sample using a machine learning model based on at least a negative marker, based on the first mass information; and
identifying a species for the input sample based on the classification result.

2. The method according to claim 1,
wherein the classifying comprises:
classifying the input sample using a positive marker and the negative marker.

3. The method according to claim 2,
wherein each of the positive marker and the negative marker is previously extracted for each of samples belonging to the similar species.

4. The method according to claim 2,
wherein the positive marker comprises mass information that frequently appears in a target species compared to an opposition species.

5. The method according to claim 2,
wherein the negative marker comprises mass information that frequently appears in an opposition species compared to a target species.

6. The method according to claim 2,
wherein each of the positive marker and the negative marker is extracted based on a bin set for a mass spectrum for each of the samples belonging to the similar species.

7. The method according to claim 6,
wherein each of the positive marker and the negative marker is expressed by a set of number of the bin in which a peak value of the mass spectrum is located.

8. The method according to claim 6,
wherein a bin partially overlaps one or more other bins.

9. The method according to claim 6,
wherein each of the positive marker and the negative marker is calculated based on frequency information of a bin in which a peak value of the mass spectrum is located.

10. The method according to claim 9,
wherein each of the positive marker and the negative marker is extracted based on a Term Frequency-Inverse Document Frequency (TF-IDF) calculation for the frequency information of the bin.

11. The method according to claim 10,
wherein the positive marker is calculated based on a math expression
$$TF - {IDF}_{bin ( i )} = \frac{{F}_{bin ( i ) , {sample}_{t}}}{{N}_{t}} \times log ( \frac{{N}_{o}}{{F}_{bin ( i ) , {sample}_{o}}} )$$
where t denotes a target species, o denotes an opposition species, Nt denotes a total number for the target species, No denotes a total number for the opposition species, and Fbin(i) denotes a count value for the i-th bin.

12. The method according to claim 11,
wherein the positive marker is set when the TF-IDF value calculated by the math expression exceeds a predetermined threshold value.

13. The method according to claim 10,
wherein the negative marker is calculated based on a math expression
$$TF - {IDF}_{bin ( i )} = \frac{{F}_{bin ( i ) , {sample}_{o}}}{{N}_{o}} \times log ( \frac{{N}_{t}}{{F}_{bin ( i ) , {sample}_{t}}} )$$
where t denotes a target species, o denotes an opposition species, Nt denotes a total number for the target species, No denotes a total number for opposition species, and . Fbin(i) denotes a count value for the i-th bin.

14. The method according to claim 13,
wherein the negative marker is set when the TF-IDF value calculated by the math expression exceeds a predetermined threshold value.

15. The method according to claim 2,
wherein each of the positive marker and the negative marker is generated as a preprocessing for extracting features for learning of the machine learning model.

16. The method according to claim 1,
wherein the classifying further comprises:
calculating a Composite Correlation Index (CCI) based on the first mass information and second mass information previously stored for each of one or more samples; and
determining a candidate for the classification based on the calculated CCI.

17. An apparatus for identifying similar species, the apparatus comprising:
a mass analyzer for extracting first mass information for an input sample; and
a classifier for classifying the input samples using a machine learning model based on at least a negative marker stored in a negative marker database, based on the first mass information,
wherein the apparatus identifies a species for the input sample based on the classification result.

18. The apparatus according to claim 17,
wherein the classifier classifies the input sample using a positive marker stored in a positive marker database and the negative marker.

19. The apparatus according to claim 18,
wherein the positive marker database and the negative marker database respectively stores the positive marker and the negative marker that are previously extracted for each of samples belonging to the similar species.

20. The apparatus according to claim 18,
wherein the apparatus further comprises:
a similarity calculator for calculating a Composite Correlation Index (CCI) based on the first mass information and second mass information for each of one or more samples previously stored in a database, and determining a candidate for the classification based on the calculated CCI.
